(19) United States
(12) Patent Application Publication
(10) Pub. No.: US 2026/0018272 A1
(43) Pub. Date: Jan. 15, 2026

(54) INFORMATION PROCESSING DEVICE, INFORMATION PROCESSING METHOD, AND COMPUTER READABLE NON-TRANSITORY STORAGE MEDIUM STORING PROGRAM

(71) Applicant: ASAHI KASEI HOMES CORPORATION, Tokyo (JP)

(72) Inventors: Shuichi OBUCHI, Tokyo (JP); Kaori ITO, Tokyo (JP); Hirotaka TOYODA, Tokyo (JP)

(73) Assignee: ASAHI KASEI HOMES CORPORATION, Tokyo (JP)

(21) Appl. No.: 19/336,872

(22) Filed: Sep. 23, 2025

Related U.S. Application Data
(63) Continuation of application No. PCT/JP2024/012802, filed on Mar. 28, 2024.

(30) Foreign Application Priority Data
Mar. 29, 2023 (JP) 2023-053507

(51) Int. Cl. G16H 20/30

(52) U.S. Cl. CPC G16H 20/30

(57) ABSTRACT

An information processing device includes an information acquisition unit configured to acquire daily activity type information, which is information indicating a daily activity type that is a type of a daily activity of a user, and daily activity frequency information, which is information indicating the frequency of the daily activity for each daily activity type, and an index calculation unit configured to calculate an index related to the daily activity by using the frequency for each daily activity type and a weight coefficient set for each daily activity type, based on the daily activity type information and the daily activity frequency information acquired by the information acquisition unit.

This application is a continuation application of International Application No. PCT/JP2024/012802, filed on Mar. 28, 2024, which claims the benefit of priority of the prior Japanese Patent Application No. 2023-053507, filed in Japan on Mar. 29, 2023, the entire contents of which are incorporated herein by reference.

### BACKGROUND OF THE INVENTION

### Field of the Invention

The present invention relates to an information processing device, an information processing method, and a computer readable non-transitory storage medium storing a program.

### Description of Related Art

A device that measures daily activities of a user for the purpose of preventing dementia is known (Patent Document 1).

### RELATED ART DOCUMENT

### Patent Document

Patent Document 1: Japanese Unexamined Patent Application, First Publication No. 2022-155611

### SUMMARY OF THE INVENTION

### Technical Problem

When predicting frailty or dementia based on daily activities, in a case where an evaluation target is biased toward either the quantity or quality of the daily activities, it is insufficient for preventing frailty or dementia.

An index that integrates the quantity and quality of the daily activities is required for the prevention of frailty or dementia. In other words, there is a demand for evaluating diversity of the daily activities.

The present invention has been made in view of the above points, and provides an information processing device, an information processing method, and a computer readable non-transitory storage medium storing a program capable of evaluating the diversity of daily activities.

### Solution to Problem

The present invention has been made in order to solve the above-described problem, and one aspect of the present invention is an information processing device including: an information acquisition unit configured to acquire daily activity type information, which is information indicating a daily activity type that is a type of a daily activity of a user, and daily activity frequency information, which is information indicating the frequency of the daily activity for each daily activity type; and an index calculation unit configured to calculate an index related to the daily activity by using the frequency for each daily activity type and a weight coefficient set for each daily activity type, based on the daily activity type information and the daily activity frequency information acquired by the information acquisition unit.

In addition, one aspect of the present invention is an information processing device including: an information acquisition unit configured to acquire daily activity type information, which is information indicating a daily activity type that is a type of a daily activity of a user, and daily activity frequency information, which is information indicating the frequency of the daily activity for each daily activity type; and an index calculation unit configured to calculate an index related to the daily activity by using the frequency for each daily activity type and a weight coefficient set for each daily activity type, based on the daily activity type information and the daily activity frequency information acquired by the information acquisition unit, in which the daily activity type includes an intellectually active daily activity and a passive and physiological daily activity, the weight coefficient set for the intellectually active daily activity is greater than the weight coefficient set for the passive and physiological daily activity, and the index calculation unit calculates the index based on the following Equation (A).

In addition, one aspect of the present invention is an information processing device including: an information acquisition unit configured to acquire daily activity type information, which is information indicating a daily activity type that is a type of a daily activity of a user, daily activity frequency information, which is information indicating the frequency of the daily activity for each daily activity type, total sedentary behavior time information, which is information indicating a length of a sedentary behavior of the user, and sedentary-interruption presence/absence information indicating whether or not the sedentary behavior is interrupted during the sedentary behavior; an index calculation unit configured to calculate an index related to the daily activity by using the frequency for each daily activity type and a weight coefficient set for each daily activity type, based on the daily activity type information and the daily activity frequency information acquired by the information acquisition unit, and configured to calculate an index related to an actual length of the sedentary behavior based on the total sedentary behavior time information and the sedentary-interruption presence/absence information acquired by the information acquisition unit; and an index derivation unit configured to obtain an index related to the daily activity including the sedentary behavior by using the index related to the daily activity and the index related to the actual length of the sedentary behavior, in which the daily activity type includes an intellectually active daily activity and a passive and physiological daily activity, and the weight coefficient set for the intellectually active daily activity is greater than the weight coefficient set for the passive and physiological daily activity.

In addition, one aspect of the present invention is an information processing method including: an information acquisition step of acquiring daily activity type information, which is information indicating a daily activity type that is a type of a daily activity of a user, and daily activity frequency information, which is information indicating the frequency of the daily activity for each daily activity type; and an index calculation step of calculating an index related to the daily activity by using the frequency for each daily activity type and a weight coefficient set for each daily activity type, based on the daily activity type information and the daily activity frequency information acquired in the information acquisition step.

In addition, one aspect of the present invention is a computer readable non-transitory storage medium storing a program causing a computer to execute: an information acquisition step of acquiring daily activity type information, which is information indicating a daily activity type that is a type of a daily activity of a user, and daily activity frequency information, which is information indicating the frequency of the daily activity for each daily activity type; and an index calculation step of calculating an index related to the daily activity by using the frequency for each daily activity type and a weight coefficient set for each daily activity type, based on the daily activity type information and the daily activity frequency information acquired in the information acquisition step.

### Advantageous Effects of Invention

According to the present invention, it is possible to evaluate the diversity of the daily activities.

### BRIEF DESCRIPTION OF THE DRAWINGS

FIG. 1 is a diagram showing an example of an outline of a configuration of an information processing system 1 according to a first embodiment.

FIG. 2 is a diagram showing an example of a daily activity type according to the embodiment of the present invention.

FIG. 3 is a diagram showing an example of a functional configuration of an information processing device 2 according to the embodiment of the present invention.

FIG. 4 is a diagram showing an example of a flow of a daily activity evaluation process in the information processing system 1 according to the embodiment of the present invention.

FIG. 5 is a diagram showing an example of a daily activity type, a frequency score, and a weight coefficient according to the embodiment of the present invention.

FIG. 6 is a diagram showing an example of a functional configuration of an information processing device 2a according to a second embodiment.

FIG. 7 is a diagram showing an example of a calculation result of an actual total sedentary time of the present embodiment.

FIG. 8 is a diagram showing an example of indices related to daily activities including a sedentary behavior derived by an index derivation unit 25 of the present embodiment.

FIG. 9 is a diagram showing a modification example of an index after sedentary correction of the present embodiment.

FIG. 10 is a diagram showing an example of a sedentary-corrected frequency score using a sedentary correction coefficient of the present embodiment.

### DETAILED DESCRIPTION OF THE INVENTION

### First Embodiment

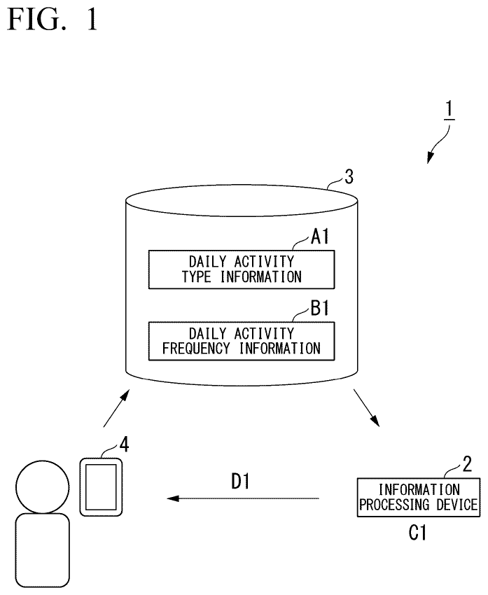

Hereinafter, embodiments of the present invention will be described in detail with reference to the drawings. FIG. 1 is a diagram showing an example of an outline of the configuration of an information processing system 1 according to a first embodiment. The information processing system 1 includes an information processing device 2, a data server 3, and a user terminal 4.

In the information processing system 1, an index C1 related to daily activities of a user is calculated. The index C1 is an index for comprehensively evaluating the quality and quantity of the daily activities of the user. In other words, the index C1 is an index for evaluating the diversity of the daily activities.

The user inputs an answer to a question about the daily activities by using the user terminal 4. The user terminal 4 is, for example, a smartphone, a tablet terminal, or a personal computer (PC). The answer input to the user terminal 4 is transmitted to the data server 3 as daily activity type information A1 and daily activity frequency information B1. The data server 3 stores the daily activity type information A1 and the daily activity frequency information B1.

The daily activity type information A1 is information indicating a daily activity type that is a type of the daily activity of the user. The daily activity frequency information B1 is information indicating the frequency of the daily activity of the user for each daily activity type.

The information processing device 2 calculates the index C1 based on the daily activity type information A1 and the daily activity frequency information B1 stored in the data server 3. The information processing device 2 evaluates a risk of frailty or dementia for the user based on the index C1. The information processing device 2 transmits an evaluation result to the user terminal 4 as evaluation result information D1.

The information processing device 2, the data server 3, and the user terminal 4 can communicate with each other (for example, wireless communication).

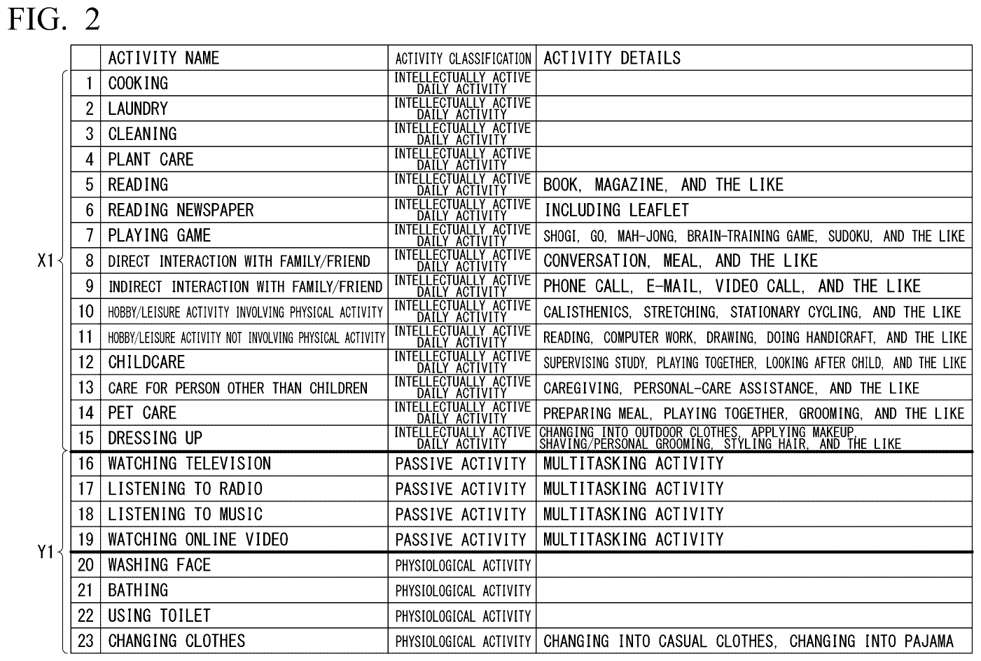

Here, the daily activity type is set in advance. FIG. 2 is a diagram showing an example of the daily activity type according to the present embodiment. In order to determine the daily activity type, for example, a factor structure for the influence on the indices C1 of a plurality of question items (daily activity types) is analyzed in advance. Based on results of the factor structure analysis, the plurality of question items are classified into a predetermined number of factors. For example, several tens to several hundreds of question items are classified into several groups based on the results of the factor structure analysis.

In the present embodiment, the daily activity types are classified into two groups in order to set a weight coefficient for calculating the index C1. In this case, for example, the daily activity types may be classified into two groups based on the results of the factor structure analysis.

In the present embodiment, the daily activity types are classified into two groups of an intellectually active daily activity and a passive and physiological daily activity. In FIG. 2, an item X1 is an item indicating an intellectually active daily activity. An item Y1 is an item indicating a passive and physiological daily activity.

The intellectually active daily activities are daily activities involving an intellectual engagement. The intellectually active daily activities include, for example, intellectual activities, creative activities, activities that requires concentration, activities involving interaction with others, activities in which a plurality of partial activities need to be performed in a specific order to complete the whole, and activities that require a plurality of activities to be performed simultaneously or in parallel.

The passive and physiological daily activities are activities that are performed passively or activities that are caused by a physiological phenomenon. The activities that are performed passively include, for example, activities that can be performed while doing another activity without concentrating on the other activity, and habitual activities that can be performed with little mental effort. The activities that are caused by a physiological phenomenon include washing the face, bathing, using the toilet, changing clothes, and the like.

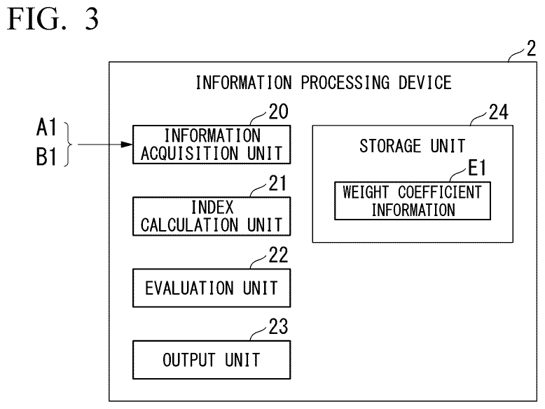

FIG. 3 is a diagram showing an example of a functional configuration of the information processing device 2 according to the present embodiment. The information processing device 2 is, for example, a computer such as a PC or a server. The information processing device 2 includes an information acquisition unit 20, an index calculation unit 21, an evaluation unit 22, an output unit 23, and a storage unit 24.

Each of these functional units is realized, for example, by a CPU loading a program read from a read only memory (ROM) into a random-access memory (RAM) and executing processing in accordance with the program. The ROM and the RAM are included in the storage unit 24.

The information acquisition unit 20 acquires the daily activity type information A1 and the daily activity frequency information B1. In the present embodiment, as an example, the information acquisition unit 20 acquires the daily activity type information A1 and the daily activity frequency information B1 from the data server 3.

The index calculation unit 21 calculates the index C1 related to the daily activity using the frequency of the daily activity for each daily activity type and a weight coefficient set for each daily activity type, based on the daily activity type information A1 and the daily activity frequency information B1 acquired by the information acquisition unit 20. The weight coefficient is set in advance for each daily activity type. Weight coefficient information E1, which is information indicating the weight coefficient, is stored in the storage unit 24 in advance.

The evaluation unit 22 evaluates the daily activity of the user based on the index C1 calculated by the index calculation unit 21. The evaluation of the daily activity includes an evaluation of signs of at least one of frailty or dementia.

The output unit 23 outputs an evaluation result by the evaluation unit 22 as evaluation result information D1. In the present embodiment, as an example, the output unit 23 transmits the evaluation result information D1 to the user terminal 4.

The storage unit 24 stores various types of information. The information stored in the storage unit 24 includes the weight coefficient information E1. The storage unit 24 is configured using a storage device such as a magnetic hard disk device or a semiconductor storage device.

The information processing device 2 may be realized as a virtual server. The functional units of the information processing device 2 may be distributed among a plurality of servers. The information processing device 2 may be realized as a cloud server.

In addition, the information processing device 2 and the data server 3 may be an integrated device. In that case, the information processing device 2 includes an information generation unit that generates the daily activity type information A1 and the daily activity frequency information B1. The daily activity type information A1 and the daily activity frequency information B1 generated by the information generation unit are stored in the storage unit 24. The information acquisition unit 20 acquires the daily activity type information A1 and the daily activity frequency information B1 from the storage unit 24.

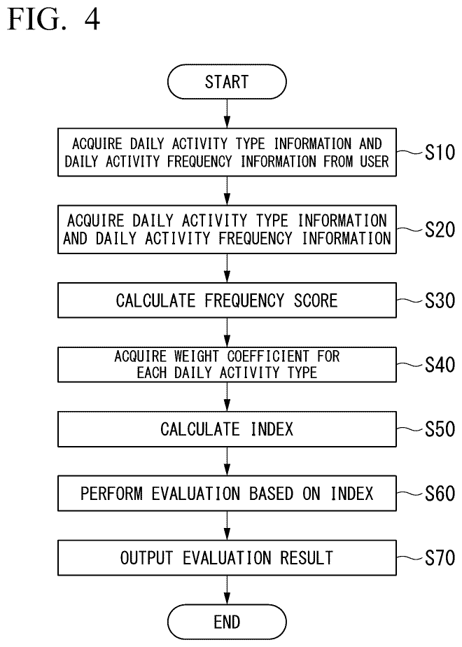

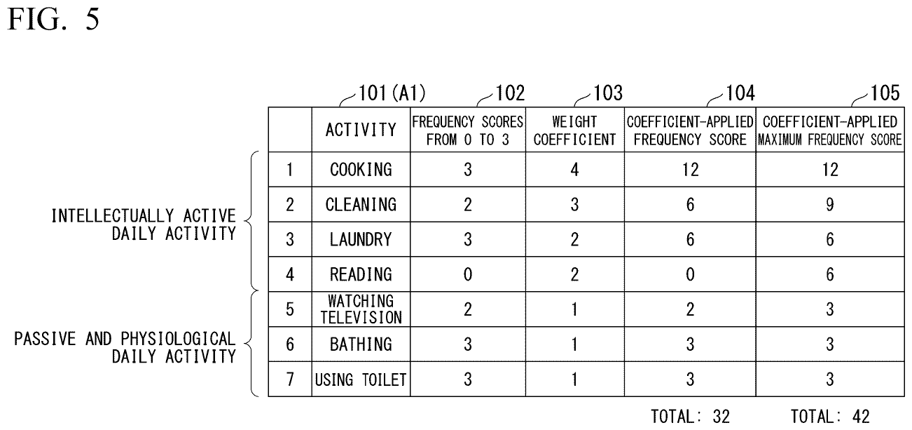

Next, a daily activity evaluation process, which is a process performed by the information processing system 1, will be described with reference to FIGS. 4 and 5. FIG. 4 is a diagram showing an example of a flow of the daily activity evaluation process in the information processing system 1 according to the present embodiment. FIG. 5 is a diagram showing an example of a daily activity type, a frequency score, and a weight coefficient according to the present embodiment. The frequency score is a score based on the frequency of the daily activity.

Step S10: The user terminal 4 acquires the daily activity type information A1 and the daily activity frequency information B1 from the user. In the present embodiment, the user answers a questionnaire via the user terminal 4. An application for answering the questionnaire is installed in advance on the user terminal 4.

In the questionnaire, the user is asked to report the daily activity frequency for each daily activity type. The content of the questionnaire is, for example, to ask the user to report the daily activity frequency for each daily activity type over a predetermined period (for example, one week) in the past. Specifically, in the questionnaire, for the question “How many times did you do the following activities during the past week?”, the user is asked to report one of four options of “0: rarely”, “1: 1 to 2 days a week”, “2: once every two days”, and “3: almost every day” for each daily activity type.

The user terminal 4 generates the daily activity type information A1 and the daily activity frequency information B1 from the answer to the input questionnaire. The frequency indicated by the daily activity frequency information B1 is calculated based on the user's answer to the question about the frequency. The process of generating the daily activity type information A1 and the daily activity frequency information B1 from the answer to the questionnaire is executed by an application installed on the user terminal 4.

An item 101 shown in FIG. 5 is an example of the daily activity type information A1. In the example of FIG. 5, the daily activity type information A1 indicates seven daily activity types of “cooking”, “cleaning”, “laundry”, “reading”, “watching television”, “bathing”, and “using toilet”.

The daily activity frequency information B1 indicates, for each daily activity type, the frequency with which the activity was performed during the past week using four stages, for example, “rarely”, “1 to 2 days a week”, “once every two days”, and “almost every day”.

As described above, in the present embodiment, as an example, the daily activity types include an intellectually active daily activity and a passive and physiological daily activity. In the example of FIG. 5, “cooking”, “cleaning”, “laundry”, and “reading” are intellectually active daily activities. In addition, “watching television”, “bathing”, and “using toilet” are passive and physiological daily activities.

The user terminal 4 transmits the generated daily activity type information A1 and daily activity frequency information B1 to the data server 3. The data server 3 stores the received daily activity type information A1 and daily activity frequency information B1.

When the data server 3 receives a new answer from the user terminal 4, the data server 3 updates the daily activity type information A1 and the daily activity frequency information B1 that have already been stored with the newly received answer. When the data server 3 receives a new answer from the user terminal 4, the data server 3 may update the daily activity type information A1 and the daily activity frequency information B1 that have already been stored by combining the newly received answer with the answer received in the past.

The process of generating the daily activity type information A1 and the daily activity frequency information B1 from the answer to the questionnaire may be executed by the data server 3 instead of the application installed on the user terminal 4. In this case, the answer to the questionnaire is transmitted from the user terminal 4 to the data server 3.

The following processes of steps S20 to S70 are executed by the information processing device 2. The information processing device 2 executes the processes of steps S20 to S70 periodically (for example, every week). When a request is received from the user terminal 4, the information processing device 2 may execute the processes of step S20 to step S70.

Step S20: The information acquisition unit 20 acquires the daily activity type information A1 and the daily activity frequency information B1 from the data server 3.

Step S30: The index calculation unit 21 calculates the frequency score based on the daily activity frequency information B1. In FIG. 5, the frequency score is shown in an item 102. The frequency scores are numbers (for example, positive integers) from 0 to 3 associated with four options in the questionnaire. Note that “0: rarely”, “1:1 to 2 days a week”, “2: once every two days”, and “3: almost every day” are converted into frequency scores of 0, 1, 2, and 3, respectively.

Step S40: The index calculation unit 21 acquires the weight coefficient for each daily activity type. The index calculation unit 21 reads out the weight coefficient information E1 from the storage unit 24. The weight coefficient information E1 is information indicating the weight coefficient for each daily activity type. For example, the weight coefficient information E1 is data in a two-dimensional table format consisting of rows and columns in which the weight coefficient is stored for each daily activity type. An item 103 shown in FIG. 5 is an example of a weight coefficient for each daily activity type. The weight coefficient may be a natural number or a real number as long as it is greater than 0. That is, the weight coefficient is a positive real number.

Here, the weight coefficient set for the intellectually active daily activity is greater than the weight coefficient set for the passive and physiological daily activity. In the item 103 shown in FIG. 5, for example, the weight coefficient set for the intellectually active daily activity is a large number equal to or greater than 2. The weight coefficients set for the passive and physiological daily activities are all 1.

For the weight coefficient set for the intellectually active daily activity, for example, the weight coefficient may be set greater for the intellectually active daily activities that are regarded as involving higher intellectual engagement, requiring greater concentration, or being more complex.

For the weight coefficient set for the passive and physiological daily activity, the weight coefficient set for the activity that is performed passively may be set to be greater than the weight coefficient set for the activity that is caused by the physiological phenomenon.

Step S50: The index calculation unit 21 calculates the index C1. Here, the index calculation unit 21 calculates the index C1 related to the daily activity using the frequency of the daily activity for each daily activity type and a weight coefficient set for each daily activity type, based on the daily activity type information A1 and the daily activity frequency information B1 acquired by the information acquisition unit 20.

Here, the index calculation unit 21 calculates a coefficient-applied frequency score by multiplying the frequency score by a weight coefficient. The coefficient-applied frequency score is a score for each daily activity type based on the frequency of the daily activity and the weight coefficient.

An item 104 shown in FIG. 5 is an example of the coefficient-applied frequency score. The maximum value of the coefficient-applied frequency score is predetermined for each daily activity type. An item 105 is the maximum value of the coefficient-applied frequency score. The maximum value of the coefficient-applied frequency score is a value obtained by multiplying the maximum value of the frequency score by the weight coefficient. In the example shown in FIG. 5, the total of the maximum values of the coefficient-applied frequency scores for the daily activity type is 42, and the total of the coefficient-applied frequency scores is 32.

In the present embodiment, the index calculation unit 21 calculates the index C1 based on the following Equation (1).

$$\begin{matrix}[ Math 1 ] & \\ Diversity score = - ( \frac{1}{log m} ) {\sum }_{i = 1}^{m} {P}_{i} ( log {P}_{j} ) & ( 1 )\end{matrix}$$

Here,
m: the number of the daily activity types,
Pᵢ: a ratio of a coefficient-applied frequency score for a certain daily activity type to a total of the maximum values of the coefficient-applied frequency scores for the daily activity types (referred to as frequency ratio), and
diversity score: the index C1.

In the example shown in FIG. 5, the value of m is 7. The frequency ratio P₁ is 12/42. The frequency ratio P₂ is 6/42. The frequency ratio P₃ is 6/42. The frequency ratio P₄ is 0/42. The frequency ratio P₅ is 2/42. The frequency ratio P₆ is 3/42. The frequency ratio P₇ is 3/42.

Equation (1) is an equation based on entropy. The diversity score (that is, the index C1) represented by Equation (1) can take values ranging from 0 to 1. A higher diversity score indicates that the number of the daily activity types included in the daily activities of the user is greater and that the daily activities are more evenly distributed across all of the preset daily activity types.

The index C1 is higher as the number of performed daily activity types is larger and the frequency of the daily activity is higher. In this sense, the quantity of the daily activity is evaluated by the index C1. In addition, the index C1 is calculated based on a weight coefficient set according to each of the daily activity types, which are classified into a plurality of types such as the intellectually active daily activity and the passive and physiological daily activity. In this sense, the quality of the daily activity is evaluated by the index C1.

On the other hand, since the maximum value of the coefficient-applied frequency score is determined in advance for each daily activity type, simply having a high frequency score for a specific daily activity type does not necessarily result in a high value of the index C1. In order to increase the value of the index C1, it is necessary to have a high frequency score for each of various daily activity types. Therefore, the diversity (quantity and quality) of the daily activities is evaluated by the index C1 calculated using Equation (1).

Step S60: The evaluation unit 22 evaluates the daily activity of the user based on the index C1 calculated by the index calculation unit 21. The evaluation unit 22 evaluates, for example, the risk of frailty or dementia based on the index C1. When the value of the index C1 is lower than a predetermined reference value for evaluating the risk of frailty, the evaluation unit 22 determines that the risk of frailty of the user is high. When the value of the index C1 is lower than a predetermined reference value for evaluating the risk of dementia, the evaluation unit 22 determines that the risk of dementia of the user is high. When the value of the index C1 is lower than a predetermined reference value for comprehensively evaluating the risk of frailty and dementia, the evaluation unit 22 may determine that the risk of frailty and dementia of the user is high.

The evaluation unit 22 may convert the magnitude of the value of the index C1 into a percentage indicating the risk of frailty based on a predetermined rule for evaluating the risk of frailty. The evaluation unit 22 may convert the magnitude of the value of the index C1 into a percentage indicating the risk of dementia based on a predetermined rule for evaluating the risk of dementia. The evaluation unit 22 may convert the magnitude of the value of the index C1 into a percentage indicating the risk of frailty and dementia based on a predetermined rule for evaluating the risk of frailty and dementia. In those cases, a predetermined rule is that the lower the value of the index C1, the higher the percentage indicating risk.

The evaluation unit 22 may evaluate the daily activity of the user based on a result of comparing the index C1 calculated in the past for the user with the newly calculated index C1. For example, when the newly calculated index C1 is lower than the index C1 calculated in the past, the evaluation unit 22 determines that the risk of frailty, dementia, or frailty and dementia is increased. When the newly calculated index C1 is higher than the index C1 calculated in the past, the evaluation unit 22 determines that the risk of frailty, dementia, or frailty and dementia is decreased.

Step S70: The output unit 23 outputs an evaluation result by the evaluation unit 22 as evaluation result information D1. In the present embodiment, the output unit 23 transmits the evaluation result information D1 to the user terminal 4. The user checks the evaluation result information D1 displayed on the user terminal 4.

The output unit 23 may output the evaluation result information DI to a device other than the user terminal 4. For example, the output unit 23 may store the evaluation result information D1 in the storage unit 24 or the data server 3. In that case, for example, when the evaluation result information D1 is stored in the storage unit 24, in a case where there is a request to transmit the evaluation result information DI from the user terminal 4, the output unit 23 transmits the evaluation result information D1 stored in the storage unit 24 to the user terminal 4. When the evaluation result information D1 is stored in the data server 3, in a case where there is a request to transmit the evaluation result information D1 from the user terminal 4, the data server 3 transmits the stored evaluation result information D1 to the user terminal 4.

In addition, the output unit 23 may transmit the evaluation result information D1 to a terminal device owned by a family of the user or a life counseling advisor. In that case, the evaluation result information D1 is suitably used, for example, by the family of the user or the life counseling advisor to give advice on the daily activity to the user. In addition, in a case where the user lives separately from his/her family, the family can use the evaluation result information D1 to check a status of the daily activity of the user from the viewpoint of prevention of one or more of frailty and dementia of the user.

In addition, the output unit 23 may output the evaluation result information D1 to each of a plurality of devices described above.

With this, the information processing system 1 ends the daily activity evaluation process.

The processes of step S60 and step S70 from the daily activity evaluation process shown in FIG. 4 may be omitted. In that case, the evaluation unit 22 and the output unit 23 may be omitted from the configuration of the information processing device 2. In addition, in this case, the index calculation unit 21 stores the calculated index C1 in the storage unit 24, for example. Alternatively, the index calculation unit 21 may output the calculated index C1 to the data server 3, the user terminal 4, or another external device.

In the present embodiment, although an example of a case where the question items of the questionnaire ask the user to report the daily activity frequency for each daily activity type during the past week has been described, the present invention is not limited to this.

The length of the predetermined period in the past may be other than one week. The length of the predetermined period may be any of 1 day to 30 days or may be 30 days or more. The question items for acquiring the frequency of the daily activity are changed as appropriate depending on the length of the predetermined period in the past.

The question items of the questionnaire are not limited to the above-described examples. The question items of the questionnaire may ask the user to report the specific number of times of the daily activity for each daily activity type during the predetermined period in the past.

In the present embodiment, an example of a case where the answer to the questionnaire is made by using the application installed on the user terminal 4 has been described, but the present invention is not limited to this. The user may answer the questionnaire by filling out a paper form on which the question items are printed. In addition, a third party may perform a medical interview on the question items for the user, and the answers from the user may be recorded by the third party.

When the daily activity frequency for each daily activity type is obtained by answering questions on paper or through a medical interview, the daily activity type information A1 and the daily activity frequency information B1 are generated by creating electronic data based on the daily activity frequency for each acquired daily activity type. The created electronic data is uploaded to the data server 3.

In addition, the daily activity type and the daily activity frequency may be acquired by using a sensor. The sensor is installed, for example, in a predetermined location in a residence where the user performs daily activities, and detects the living activities of the user. The sensor is, for example, one or more of a human detection sensor, a camera, and the like. In addition, the sensor may be installed in an appliance provided in a residence or a door or the like, and may acquire the daily activity frequency for a specific daily activity type. The sensor may be a wearable terminal that is worn on a body of the user.

In a case where the daily activity type and the daily activity frequency are acquired by using the sensor, for example, the information acquired by the sensor is transmitted to the data server 3. The daily activity type information A1 and the daily activity frequency information B1 are generated in the data server 3 based on the information transmitted from the sensor.

In this case, the data server 3 discriminates the daily activity type and the daily activity frequency by analyzing the information acquired by the sensor. The data server 3 discriminates the daily activity type and the daily activity frequency based on, for example, machine learning or pattern matching. The data server 3 discriminates the daily activity type and the daily activity frequency from, for example, a detection result of the human detection sensor or an image captured by the camera, based on machine learning or pattern matching.

Whether a certain activity is classified as an intellectually active daily activity or as a passive and physiological daily activity may be determined according to whether the activity is being performed with concentrated attention. Therefore, even in a case of the same daily activity type, it may be classified as an intellectually active daily activity or as a passive and physiological daily activity depending on whether the daily activity is determined as being performed with concentrated attention.

For example, in a case of reading a newspaper, the degree of concentration is different between reading the newspaper with interest and reading the newspaper while doing something else. The activity of reading the newspaper with interest is classified as an intellectually active daily activity, and the activity of reading the newspaper while doing something else is classified as a passive activity.

In addition, the degree of concentration may be different even in a case where the activities themselves are the same. For example, in both “dressing up” and “changing clothes”, the underlying activity of putting on garments is the same. However, while the dressing up is performed with conscious attention to the appearance, the changing of clothes may be performed while thinking about other things. Therefore, it is considered that the degree of concentration is different between the dressing up and the changing of clothes. The dressing up is classified as an intellectually active daily activity, and the changing of clothes is classified as a passive activity.

The determination of whether a certain activity is being performed with concentrated attention is performed, for example, based on a detection result acquired by a sensor. For example, based on machine learning or pattern matching, it is determined whether a certain activity is being performed with concentrated attention from a detection result of the human detection sensor, an image captured by the camera, or the like. For the daily activity determined as being performed with concentrated attention, the weight coefficient is set to be greater than that in a case where the daily activity is not determined as being performed with concentrated attention.

In addition, in a case where a certain activity is performed with concentrated attention, the degree of concentration may be determined. In this case, the greater the determined degree of concentration, the greater the weight coefficient is set.

In the present embodiment, although an example of a case where the weight coefficient information E1 is information indicating a weight coefficient for each daily activity type has been described, the present invention is not limited to this. The weight coefficient information E1 may be information indicating a weight coefficient for each classification of the daily activity type. For example, the weight coefficient information E1 may indicate a weight coefficient for each of the intellectually active daily activity and the passive and physiological daily activity. In this case, for example, all the weight coefficients for the intellectually active daily activities are set to 2, and all the weight coefficients for the passive and physiological daily activities are set to 1.

In another example, the intellectually active daily activities may be further classified into an activity that is regarded as involving higher intellectual engagement, an activity that is regarded as requiring greater concentration, and an activity that is regarded as being more complex, and a weight coefficient may be set for each of the activities.

In another example, the passive and physiological daily activities may be further classified into an activity that is performed passively and an activity that is caused by a physiological phenomenon, and a weight coefficient may be set for each of the activities. In this case, for example, the weight coefficient for the activity that is performed passively is set to be greater than the weight coefficient for the activity caused by the physiological phenomenon.

In addition, the weight coefficient may be set for each group of the daily activity types classified based on the analysis result of the factor structure.

In the present embodiment, although an example of a case where the weight coefficient set for the intellectually active daily activity is greater than the weight coefficient set for the passive and physiological daily activity has been described, the present invention is not limited to this. The weight coefficient set for the intellectually active daily activity may be the same as the weight coefficient set for the passive and physiological daily activity or may be smaller than the weight coefficient.

Note that it is considered that it is desirable to have diversity in the daily activities in order to prevent frailty or dementia. Therefore, in order to use the index C1 to evaluate the risk of frailty or dementia, it is preferable that the weight coefficient set for the intellectually active daily activity is set to be greater than the weight coefficient set for the passive and physiological daily activity.

The index calculation unit 21 may calculate the index C1 based on the following Equation (2) instead of Equation (1) described above.

$$\begin{matrix}[ Math 2 ] & \\ Diversity score = {{\sum }_{i = 1}}^{m} Pi & ( 2 )\end{matrix}$$

In Equation (2), the meanings of m, Pᵢ, and diversity score are the same as those in Equation (1).

As described above, the information processing device 2 according to the present embodiment includes the information acquisition unit 20 and the index calculation unit 21.

The information acquisition unit 20 acquires the daily activity type information A1, which is information indicating a daily activity type that is a type of a daily activity of the user, and the daily activity frequency information B1, which is information indicating the frequency of the daily activity for each daily activity type.

The index calculation unit 21 calculates the index C1 related to the daily activity of the user using the frequency for each daily activity type and the weight coefficient set for each daily activity type, based on the daily activity type information A1 and the daily activity frequency information B1 acquired by the information acquisition unit 20.

With this configuration, in the information processing device 2 according to the present embodiment, the index C1 related to the daily activity of the user can be calculated by using the frequency for each daily activity type and the weight coefficient set for each daily activity type, thereby enabling evaluation of the diversity of the daily activities. As described in the embodiment, the calculated index C1 can be used to evaluate the risk of frailty and dementia of the user.

### Second Embodiment

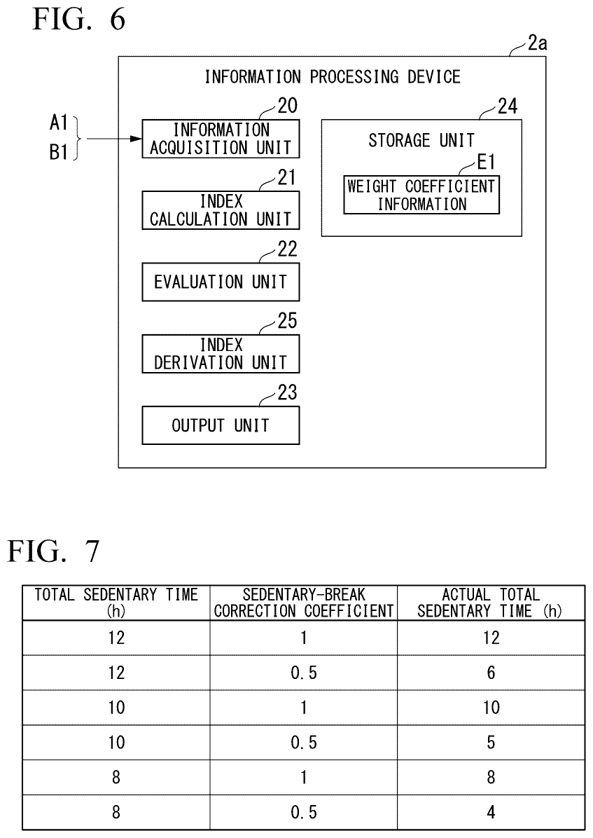

FIG. 6 is a diagram showing an example of a functional configuration of an information processing device 2a according to a second embodiment. The information processing device 2a is different from the information processing device 2 exemplified in the first embodiment in that an index derivation unit 25 is added.

In addition, the information processing device 2a is different from the information processing device 2 in that an index related to the daily activity is calculated by taking into account information about a sedentary behavior, such as whether or not the daily activity of the user is an activity in a sitting posture (hereinafter, also referred to as a sedentary behavior) and the extent or aspect in which the sedentary behavior is performed. In the following description, the index related to the daily activity in the present embodiment is also referred to as an index C1a.

The same reference numerals are assigned to the same configurations and operations as those of the information processing device 2 of the first embodiment, and the description thereof will be omitted.

In recent years, the risks of prolonged sitting in daily activities and the need for countermeasures have been pointed out. It has been pointed out that, in a case where such a sedentary behavior continues, it is possible to reduce the risk of dementia or frailty by performing an operation including standing (for example, an operation of standing up on the spot or moving around) once or more at a certain time interval (for example, 30 minutes). For example, in a case where a daily activity such as reading or watching television is performed in a sitting posture, it is recommended to stand up about once every 30 minutes in order to reduce the risk of dementia or frailty.

The information processing device 2a according to the present embodiment calculates the index C1a related to the daily activity based on information about the sedentary behavior of the user. Hereinafter, a specific example of the configuration of the information processing device 2a will be described.

### (1) Calculation of Index Based on Actual Total Sedentary Time

As described above, the information acquisition unit 20 acquires the daily activity type information A1 and the daily activity frequency information B1. In the present embodiment, as an example, the information acquisition unit 20 acquires the daily activity type information A1 and the daily activity frequency information B1 from the data server 3.

The daily activity frequency information B1 of the present embodiment includes information indicating an extent of the sedentary behavior. The degree of the sedentary behavior refers to a continuation time of the sedentary behavior, whether or not the sedentary behavior is interrupted during the sedentary behavior, the frequency of the sedentary interruptions during the sedentary behavior, or the like.

In addition, the frequency of the sedentary interruptions during the sedentary behavior refers to how often a sitting posture is temporarily resolved by standing up or the like during the sedentary behavior. In the following description, the sedentary interruption during the sedentary behavior is also referred to as a sedentary break.

As an example, for each type of activity indicated by the daily activity type information A1, the daily activity frequency information B1 includes information indicating whether the activity is performed in a posture (for example, a standing posture) other than a sitting posture or performed in a sitting posture, and includes, when the activity is performed in a sitting posture, information indicating a continuation time of the sedentary behavior and the frequency of sedentary interruptions during the sedentary behavior.

The information acquisition unit 20 acquires the continuation time (for example, the total sedentary time) of the sedentary behavior indicated by the daily activity frequency information B1 and the presence or absence of the sedentary break.

The index calculation unit 21 calculates a sedentary-break correction coefficient based on the presence or absence of the sedentary break. As an example, the index calculation unit 21 sets the sedentary-break correction coefficient to “1” in a case where there is a sedentary break, and sets the sedentary-break correction coefficient to “0.5” in a case where there is no sedentary break.

The index calculation unit 21 calculates an actual total sedentary time from the total sedentary time and the sedentary-break correction coefficient based on Equation (3) described below.

$$Actual total sedentary time = total sedentary time \times sendentary - break correction coefficient \qquad ( 3 )$$

In the following description, the actual total sedentary time is also referred to as an index (or an index C11) related to the actual length of the sedentary behavior.

FIG. 7 is a diagram showing an example of a calculation result of the actual total sedentary time of the present embodiment. For example, in a case where the total sedentary time is 12 hours and the sedentary-break correction coefficient is 1 (that is, in a case where there is a sedentary break), the index calculation unit 21 calculates the actual total sedentary time (index C11) as 12 hours. Similarly, for example, in a case where the total sedentary time is 12 hours and the sedentary-break correction coefficient is 0.5 (that is, in a case where there is no sedentary break), the index calculation unit 21 calculates the actual total sedentary time (index C11) as 6 hours.

In the above-described example, the index calculation unit 21 calculates the sedentary-break correction coefficient depending on the presence or absence of the sedentary break, but the present invention is not limited to this. For example, the index calculation unit 21 may calculate the sedentary-break correction coefficient based on the frequency of occurrence of the sedentary break. As an example, in a case where the frequency of occurrence of the sedentary break is once or more every 30 minutes while the sedentary behavior is continuing, the index calculation unit 21 calculates the sedentary-break correction coefficient as 1 (that is, there is a sedentary break). In a case where the frequency of occurrence of the sedentary break is less than once every 30 minutes while the sedentary behavior is continuing, the index calculation unit 21 calculates the sedentary-break correction coefficient as 0.5 (that is, there is no sedentary break).

That is, the information acquisition unit 20 further acquires total sedentary behavior time information, which is information indicating the length of the sedentary behavior of the user, and sedentary-interruption presence/absence information indicating whether or not the sedentary behavior is interrupted during the sedentary behavior.

The index calculation unit 21 further calculates an index C11 related to an actual length of the sedentary behavior based on the total sedentary behavior time information and the sedentary-interruption presence/absence information acquired by the information acquisition unit 20.

In addition, the index C11 related to the actual length of the sedentary behavior is calculated based on a sedentary-break correction coefficient set based on the total sedentary behavior time information and the sedentary-interruption presence/absence information.

Further, the index derivation unit 25 obtains an index related to the daily activity including the sedentary behavior by using the index C1 related to the daily activity and the index C11 related to the actual length of the sedentary behavior.

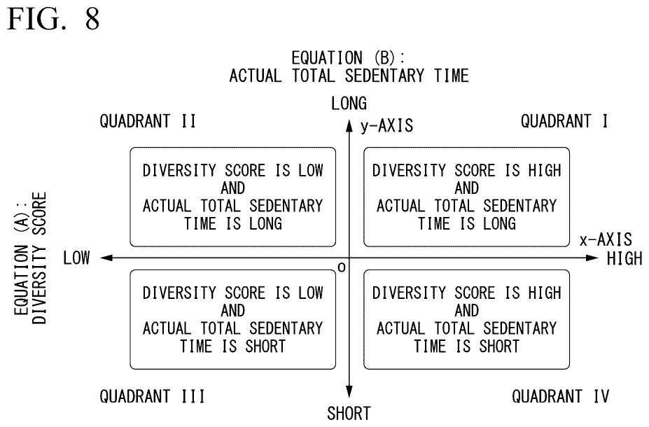

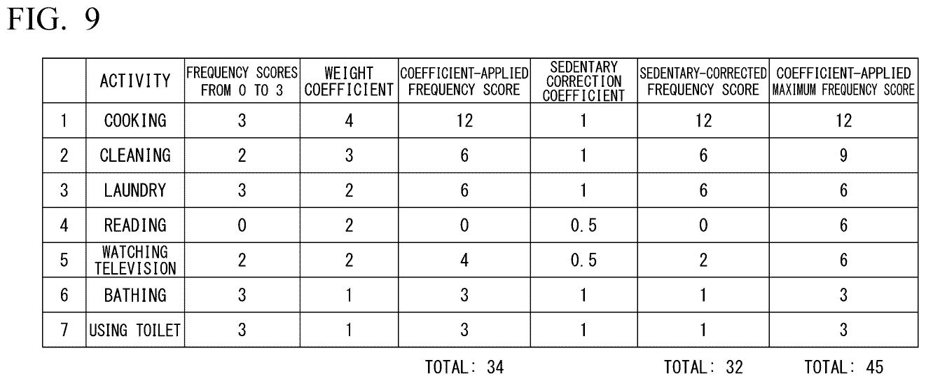

FIG. 8 is a diagram showing an example of indices related to daily activities including the sedentary behavior derived by the index derivation unit 25 of the present embodiment. In the figure, a horizontal axis (x-axis) indicates the index C1 (for example, the diversity score obtained in the first embodiment) related to the daily activity, and a vertical axis (y-axis) indicates the index C11 (for example, the actual total sedentary time) related to the actual length of the sedentary behavior.

In the figure, the evaluation is the best when the diversity score is high and the actual total sedentary time is short (quadrant IV in the figure). In addition, the evaluation is the worst when the diversity score is low and the actual total sedentary time is long (quadrant II in the figure).

By expressing the indices as in the figure, even in a case where the units of the diversity score and the actual total sedentary time are different from each other, the goodness or badness of the two can be expressed as a distance from the origin O.

The index derivation unit 25 may normalize the index C1 related to the daily activity and the index C11 related to the actual length of the sedentary behavior relative to each other. For example, the index derivation unit 25 normalizes the two indices by projecting the index C11 related to the actual length of the sedentary behavior onto the axis of the index C1 related to the daily activity, taking into account the contribution of the index C11. With the index derivation unit 25 configured in this way, the indices can be represented by one axis.

That is, the information acquisition unit 20 further acquires a sedentary correction coefficient, which is information indicating a correction coefficient based on the sedentary behavior of the user for each daily activity type. The index calculation unit 21 calculates the index based on Equation (1) described above. Note that the meanings of the parameters of Equation (1) are as follows.

Here,
m: the number of the daily activity types,
Pi: a ratio of a score (ii) for a certain daily activity type to a total (i) for the daily activity types, the total being a sum of maximum values of scores for the respective daily activity types based on the frequency and the weight coefficient, in which either both (i) and (ii), or only (ii), is corrected by the sedentary correction coefficient, and
diversity score: the index C1a.

FIG. 9 is a diagram showing a modification example of an index after sedentary correction of the present embodiment. The index calculation unit 21 calculates a sedentary-corrected frequency score using the sedentary correction coefficient instead of the sedentary-break correction coefficient described above. In this example, the index calculation unit 21 calculates a sedentary-corrected frequency score by multiplying the frequency score before the sedentary correction (for example, the index C1) by the sedentary correction coefficient. The sedentary correction coefficient is a coefficient set in advance based on the daily activity of the user.

As shown in the figure, an integrated value (for example, a total of 32) of the sedentary-corrected frequency scores is a value smaller than an integrated value (for example, a total of 34) of the coefficient-applied frequency scores before the sedentary correction. This indicates that the sedentary correction coefficient (for example, 0.5) acts as an element (in other words, a penalty) that reduces the diversity score.

The user's sedentary-corrected frequency score can be evaluated by evaluating the integrated value (for example, a total of 32) of the sedentary-corrected frequency scores relative to the maximum frequency scores (that is, the full score, for example, a total of 45) after application of the sedentary correction coefficient (for example, calculating the ratio as numerator 32/denominator 45).

The above-described calculation process by the index calculation unit 21 is an example, and the present invention is not limited to this. The index calculation unit 21 may calculate the sedentary-corrected frequency score using various combinations of the indices, weight coefficients, and correction coefficients.

The index calculation unit 21 may calculate the index C1 based on the above-described Equation (2) instead of the above-described Equation (1), as in the first embodiment.

### (2) Modification Example: Case of Calculating Diversity Score Based on Index of Sedentary Behavior

In the above-described example, the diversity score (for example, the index C1a) is calculated based on the frequency score before the sedentary correction (for example, the index C1) and the sedentary-corrected frequency score using the sedentary-break correction coefficient (or the sedentary correction coefficient), but the present invention is not limited to this.

The index calculation unit 21 may calculate the diversity score (for example, the index C1a) based only on the sedentary-corrected frequency score using the sedentary-break correction coefficient (or the sedentary correction coefficient) without using the frequency score before the sedentary correction (for example, the index C1).

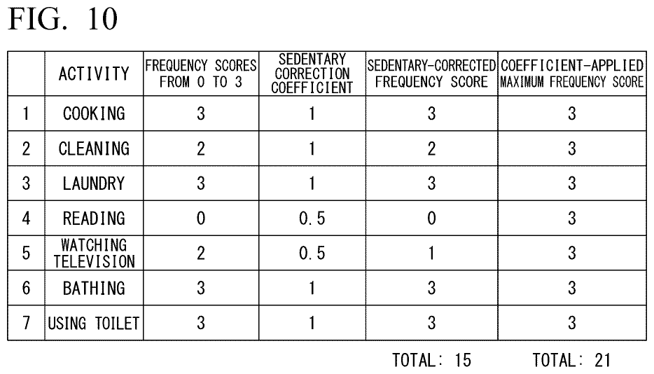

That is, the weight coefficient set for each daily activity type includes a sedentary correction coefficient set based on the extent of the sedentary behavior. FIG. 10 is a diagram showing an example of a sedentary-corrected frequency score using the sedentary correction coefficient of the present embodiment.

The index calculation unit 21 calculates the index based on Equation (1) described above.

Note that the meanings of the parameters of Equation (1) are as follows.
m: the number of the daily activity types,
Pi: a ratio of a score obtained by a certain daily activity type and the sedentary correction coefficient to a total for the daily activity types, the total being the sum of maximum values of scores for the respective daily activity types based on the frequency, and
diversity score: the index C1a.

As described above, with the information processing device 2a according to the second embodiment, it is possible to calculate the diversity score in consideration of the risk of prolonged sitting n daily activities, that is, the risk of the sedentary behavior. By using three types of diversity scores in consideration of the risk of such a sedentary behavior, it is possible to more accurately evaluate the risk of dementia or frailty, leading to a reduction in the risk.

Parts of the information processing device 2, the information processing device 2a, and the data server 3 in the above-described embodiments, for example, the information acquisition unit 20, the index calculation unit 21, the evaluation unit 22, the index derivation unit 25, and the output unit 23 may be realized by a computer. In this case, the control function may be realized by recording a program for realizing the control function on a computer readable recording medium and reading and executing the program recorded on the recording medium into a computer system. The term “computer system” referred to here is a computer system incorporated in the information processing device 2 and the data server 3, and includes hardware such as an operating system (OS) and peripheral devices. In addition, the term “computer readable recording medium” refers to a portable medium such as a flexible disk, a magneto-optical disk, a read only memory (ROM), or a compact disc-read only memory (CD-ROM), or a storage medium such as a hard disk built in a computer system. Further, the term “computer readable recording medium” may also include a medium that dynamically holds the program for a short time, such as a communication line in a case where the program is transmitted via a network such as the Internet or a communication channel such as a telephone line, and a medium that holds the program for a certain time, such as a volatile memory inside the computer system as the server or the client in that case. In addition, the program may be a program realizing some of the functions described above, and, further, the functions described above may be realizable by combining with the program previously recorded in the computer system.

In addition, a part or all of the information processing device 2, the information processing device 2a, and the data server 3 in the above-described embodiments may be realized as an integrated circuit such as a large-scale integration (LSI). The functional blocks of the information processing device 2 and the data server 3 may be individually implemented as a processor, or may be partially or entirely integrated into a processor. In addition, a method for the integrated circuit is not limited to the LSI, and may be realized by a dedicated circuit or a general-purpose processor. In addition, when a technology for the integrated circuit is introduced to replace the LSI as a semiconductor technology advances, an integrated circuit based on the technology may be used.

The embodiments of the present invention have been described in detail above with reference to the drawings, but the specific configurations are not limited to those described above, and various design changes, and the like are possible within the scope that does not deviate from the gist of the present invention.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### REFERENCE SIGNS LIST

2, 2a Information processing device
20 Information acquisition unit
21 Index calculation unit
25 Index derivation unit
A1 Daily activity type information
B1 Daily activity frequency information
C1 Index
E1 Weight coefficient information

What is claimed is:

1. An information processing device comprising:
an information acquisition unit configured to acquire daily activity type information, which is information indicating a daily activity type that is a type of a daily activity of a user, and daily activity frequency information, which is information indicating a frequency of the daily activity for each daily activity type; and
an index calculation unit configured to calculate an index related to the daily activity by using the frequency for each daily activity type and a weight coefficient set for each daily activity type, based on the daily activity type information and the daily activity frequency information acquired by the information acquisition unit.

2. The information processing device according to claim 1,
wherein the daily activity type includes an intellectually active daily activity and a passive and physiological daily activity, and
the weight coefficient set for the intellectually active daily activity is greater than the weight coefficient set for the passive and physiological daily activity.

3. The information processing device according to claim 1,
wherein the index calculation unit calculates the index based on the following Equation (A),
[Math 1]
where,
m: the number of the daily activity types,
Pᵢ: a ratio of a score for a certain daily activity type to a total for the daily activity types, the total being a sum of maximum values of scores for the respective daily activity types based on the frequency and the weight coefficient, and
diversity score: the index:
$$Diversity score = - ( \frac{1}{log m} ) {\sum }_{i = 1}^{m} {P}_{i} ( log {P}_{i} ) . \qquad ( A )$$

4. An information processing device comprising:
an information acquisition unit configured to acquire daily activity type information, which is information indicating a daily activity type that is a type of a daily activity of a user, and daily activity frequency information, which is information indicating a frequency of the daily activity for each daily activity type; and
an index calculation unit configured to calculate an index related to the daily activity by using the frequency for each daily activity type and a weight coefficient set for each daily activity type, based on the daily activity type information and the daily activity frequency information acquired by the information acquisition unit,
wherein the daily activity type includes an intellectually active daily activity and a passive and physiological daily activity,
the weight coefficient set for the intellectually active daily activity is greater than the weight coefficient set for the passive and physiological daily activity, and
the index calculation unit calculates the index based on the following Equation (A),
[Math 2]
where,
m: the number of the daily activity types,
Pᵢ: a ratio of a score for a certain daily activity type to a total for the daily activity types, the total being a sum of maximum values of scores for the respective daily activity types based on the frequency and the weight coefficient, and diversity score: the index:
$$Diversity score = - ( \frac{1}{log m} ) {\sum }_{i = 1}^{m} {P}_{i} ( log {P}_{i} ) . \qquad ( A )$$

5. The information processing device according to claim 1,
wherein the information acquisition unit further acquires total sedentary behavior time information, which is information indicating a length of a sedentary behavior of the user, and sedentary-interruption presence/absence information indicating whether or not the sedentary behavior is interrupted during the sedentary behavior,
the index calculation unit further calculates an index related to an actual length of the sedentary behavior based on the total sedentary behavior time information and the sedentary-interruption presence/absence information acquired by the information acquisition unit, and
the information processing device further comprises an index derivation unit configured to obtain an index related to the daily activity including the sedentary behavior by using the index related to the daily activity and the index related to the actual length of the sedentary behavior.

6. An information processing device comprising:
an information acquisition unit configured to acquire daily activity type information, which is information indicating a daily activity type that is a type of a daily activity of a user, daily activity frequency information, which is information indicating a frequency of the daily activity for each daily activity type, total sedentary behavior time information, which is information indicating a length of a sedentary behavior of the user, and sedentary-interruption presence/absence information indicating whether or not the sedentary behavior is interrupted during the sedentary behavior;
an index calculation unit configured to calculate an index related to the daily activity by using the frequency for each daily activity type and a weight coefficient set for each daily activity type, based on the daily activity type information and the daily activity frequency information acquired by the information acquisition unit, and configured to calculate an index related to an actual length of the sedentary behavior based on the total sedentary behavior time information and the sedentary-interruption presence/absence information acquired by the information acquisition unit; and
an index derivation unit configured to obtain an index related to the daily activity including the sedentary behavior by using the index related to the daily activity and the index related to the actual length of the sedentary behavior,
wherein the daily activity type includes an intellectually active daily activity and a passive and physiological daily activity, and
the weight coefficient set for the intellectually active daily activity is greater than the weight coefficient set for the passive and physiological daily activity.

7. The information processing device according to claim 6,
wherein the index related to the actual length of the sedentary behavior is calculated based on a sedentary-break correction coefficient set based on the total sedentary behavior time information and the sedentary-interruption presence/absence information.

8. The information processing device according to claim 1,
wherein the information acquisition unit further acquires a sedentary correction coefficient that is information indicating a correction coefficient based on a sedentary behavior of the user for each daily activity type, and
the index calculation unit calculates the index based on the following Equation (A),
[Math 3]
where,
m: the number of the daily activity types,
Pi: a ratio of a score (ii) for a certain daily activity type to a total (i) for the daily activity types, the total being a sum of maximum values of scores for the respective daily activity types based on the frequency and the weight coefficient, in which either both (i) and (ii), or only (ii), is corrected by the sedentary correction coefficient, and
diversity score: the index:
$$Diversity score = - ( \frac{1}{log m} ) {\sum }_{i = 1}^{m} {P}_{i} ( log {P}_{i} ) . \qquad ( A )$$

9. The information processing device according to claim 1,
wherein the weight coefficient set for each daily activity type includes a sedentary correction coefficient set based on an extent of a sedentary behavior.

10. The information processing device according to claim 8,
wherein the index calculation unit calculates the index based on the following Equation (A),
[Math 4]
where,
m: the number of the daily activity types,
Pi: a ratio of a score obtained by a certain daily activity type and the sedentary correction coefficient to a total for the daily activity types, the total being a sum of maximum values of scores for the respective daily activity types based on the frequency, and
diversity score: the index:
$$Diversity score = - ( \frac{1}{log m} ) {\sum }_{i = 1}^{m} {P}_{i} ( log {P}_{i} ) . \qquad ( A )$$

11. The information processing device according to claim 1,
wherein the frequency is calculated based on a user's answer to a question about the frequency.

12. An information processing method comprising:
an information acquisition step of acquiring daily activity type information, which is information indicating a daily activity type that is a type of a daily activity of a user, and daily activity frequency information, which is information indicating a frequency of the daily activity for each daily activity type; and
an index calculation step of calculating an index related to the daily activity by using the frequency for each daily activity type and a weight coefficient set for each daily activity type, based on the daily activity type information and the daily activity frequency information acquired in the information acquisition step.

13. A computer readable non-transitory storage medium storing a program causing a computer to execute:
an information acquisition step of acquiring daily activity type information, which is information indicating a daily activity type that is a type of a daily activity of a user, and daily activity frequency information, which is information indicating a frequency of the daily activity for each daily activity type; and
an index calculation step of calculating an index related to the daily activity by using the frequency for each daily activity type and a weight coefficient set for each daily activity type, based on the daily activity type information and the daily activity frequency information acquired in the information acquisition step.
